(19) United States
(12) Patent Application Publication
(10) Pub. No.: US 2025/0299840 A1
(43) Pub. Date: Sep. 25, 2025

(54) SYSTEM AND METHOD FOR REMOTE MONITORING FOR AN AMBULATORY PATIENT

(71) Applicant: Caretuner Inc., Wynnewood, PA (US)

(72) Inventor: Eyal Bartfeld, Mazkeret Batia (IL)

(73) Assignee: CareTuner Inc., Wynnewood, PA (US)

(21) Appl. No.: 19/085,203

(22) Filed: Mar. 20, 2025

Related U.S. Application Data
(60) Provisional application No. 63/569,219, filed on Mar. 25, 2024.

(51) Int. Cl. G16H 80/00; G08B 21/02; G16H 10/60; G16H 40/67

(52) U.S. Cl. CPC G16H 80/00; G08B 21/02; G16H 10/60; G16H 40/67

(57) ABSTRACT

Systems and methods for remote monitoring for an ambulatory patient, including: receiving, by a processor of a mobile computing device, a database of identification data for a plurality of objects associated with at least one event by the patient, engaging, by the mobile computing device, at least one object of the plurality of objects, verifying, by the processor of the mobile computing device, the identification data for the at least one object with the received database, and registering, by the processor of the mobile computing device, an event by the patient that is associated with the engaged object.

### CROSS-REFERENCE TO RELATED APPLICATIONS

This application claims the benefit of U.S. Provisional Patent Application No. 63/569,219, filed Mar. 25, 2024, which is hereby incorporated by reference.

### FIELD OF THE INVENTION

The present invention relates to medical care for ambulatory patients with data collection using external triggers. More particularly, the present invention relates to systems and methods for remote monitoring of ambulatory patients.

### BACKGROUND OF THE INVENTION

Over the course of time, the technology for monitoring patients quickly developed with a variety of solutions for the patients as well as for their physicians. For example, for many medical conditions many devices can be found that measure health data and can automatically deliver that data to a remote storage, making the data available for later review by stakeholders.

Some health data items cannot in some cases be measured by devices while the monitoring devices also cannot be carried by patients everywhere they go. Such data items can include mood, pain level, pain location, epileptic seizure, headache, migraine attack, menstrual cycle, taking medication, medication side effects and more. These data items can be important and relevant for the assessment of patient's condition and for planning a future therapy. Current methods for collecting such data can include relying on patients' memory, or having the patient write the data in a paper or electronic diary.

However, often, a patient who is suffering pain, anxiety or after a migraine or a seizure attack are compromised, and it can be difficult for them to find and operate a patient diary and/or manually enter the event data. Entering data in a timely manner can be important but health or medical conditions, especially for chronically ill patients, can prevent these patients from doing so, thus reducing the accuracy and utility of overall medical care.

Thus, it can be advantageous that the process of health or wellness data entry that cannot be measured by electronic devices be made easier, immediate and with as little as possible user effort because users are typically compromised in these circumstances.

### SUMMARY OF THE INVENTION

An aspect of the invention described herein provides an apparatus and a method for digitally documenting medical, health or wellness events.

There is thus provided, in accordance with some embodiments of the invention, a method of remote monitoring for an ambulatory patient, including: receiving, by a processor of a mobile computing device, a database of identification data for a plurality of objects associated with at least one event by the patient, engaging, by the mobile computing device, at least one object of the plurality of objects, verifying, by the processor of the mobile computing device, the identification data for the at least one object with the received database, and registering, by the processor of the mobile computing device, an event by the patient that is associated with the engaged object.

In some embodiments, registering the event includes sending an alert to a medical sever in communication with the mobile computing device to register the event for that patient. In some embodiments, engaging the at least one object includes determining the identification data with a time stamp.

In some embodiments, engaging the at least one object is carried out by a wireless communication module of the mobile computing device. In some embodiments, engaging the at least one object is carried out by an imager of the mobile computing device. In some embodiments, a new object may be recorded by the imager of the mobile computing device.

In some embodiments, an alert is issued when a rate of registering events exceeds a predefined threshold, and a request is sent for a new prescription of a medication, wherein the registered event is for taking the medication.

In some embodiments, a time delay is applied, by the processor of the mobile computing device, between consecutive registering of events. In some embodiments, a unique identification code is generated, and an event is associated to a new object by the generated unique identification code, by the processor of the mobile computing device.

In some embodiments, identification data for a plurality of objects is stored on a dedicated server, and the identification data is recovered from the dedicated server, when the mobile computing device is not available.

There is thus provided, in accordance with some embodiments of the invention, a system for remote monitoring for an ambulatory patient, including: a remote server with a database of identification data for a plurality of objects associated with at least one event by the patient, and a processor of a mobile computing device, in communication with the remote server, wherein the processor is configured to: verify the identification data for at least one object of the plurality of objects with the received database, when the computing device engages the at least one object of the plurality of objects, and register an event by the patient that is associated with the engaged object.

In some embodiments, the processor is further configured to send an alert to a medical sever in communication with the mobile computing device to register the event for that patient. In some embodiments, the processor is further configured to determine the identification data with a time stamp, when the computing device engages at least one object. In some embodiments, the mobile computing device comprises a wireless communication module to engage the at least one object. In some embodiments, the mobile computing device comprises an imager to engage the at least one object. In some embodiments, the processor is further configured to record a new object by the imager of the mobile computing device.

In some embodiments, the processor is further configured to: issue an alert when a rate of registering events exceeds a predefined threshold, and send a request for a new prescription of a medication, wherein the registered event is for taking the medication. In some embodiments, processor is further configured to: generate a unique identification code, and associate an event to a new object by the generated unique identification code.

In some embodiments, the processor is further configured to: store identification data for a plurality of objects on the remote server, and recover the identification data from the remote server, when the mobile computing device is not available.

There is thus provided, in accordance with some embodiments of the invention, a system for remote monitoring for an ambulatory patient, including: a processor of a mobile computing device, including: a database of identification data for a plurality of objects associated with at least one event by the patient, wherein the processor is configured to: verify the identification data for at least one object of the plurality of objects with the database, when the computing device engages the at least one object of the plurality of objects, and register an event by the patient that is associated with the at least one object.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 shows a block diagram of a computing device, according to some embodiments of the invention;

FIG. 2A shows a block diagram for a system for remote monitoring for an ambulatory patient with a dedicated database on a remote server, according to some embodiments of the invention;

FIG. 2B shows a block diagram for a system for remote monitoring for an ambulatory patient with a dedicated database on a mobile device, according to some embodiments of the invention;

FIG. 3A shows a flowchart for associating an event with NFC tags, according to some embodiments of the invention;

FIG. 3B shows a flowchart for reporting an event with NFC tags, according to some embodiments of the invention;

FIG. 4A shows a flowchart for associating an event with a QR code, according to some embodiments of the invention;

FIG. 4B shows a flowchart for reporting an event with QR codes, according to some embodiments of the invention;

FIG. 5 shows a flowchart for associating an event with a predefined object, according to some embodiments of the invention; and

FIG. 6, which shows a flowchart for a method of remote monitoring for an ambulatory patient, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details.

In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items.

Unless explicitly stated, the method embodiments described herein are not constrained to a particular order or sequence. Additionally, some of the described method embodiments or elements thereof may occur or be performed simultaneously, at the same point in time, or concurrently.

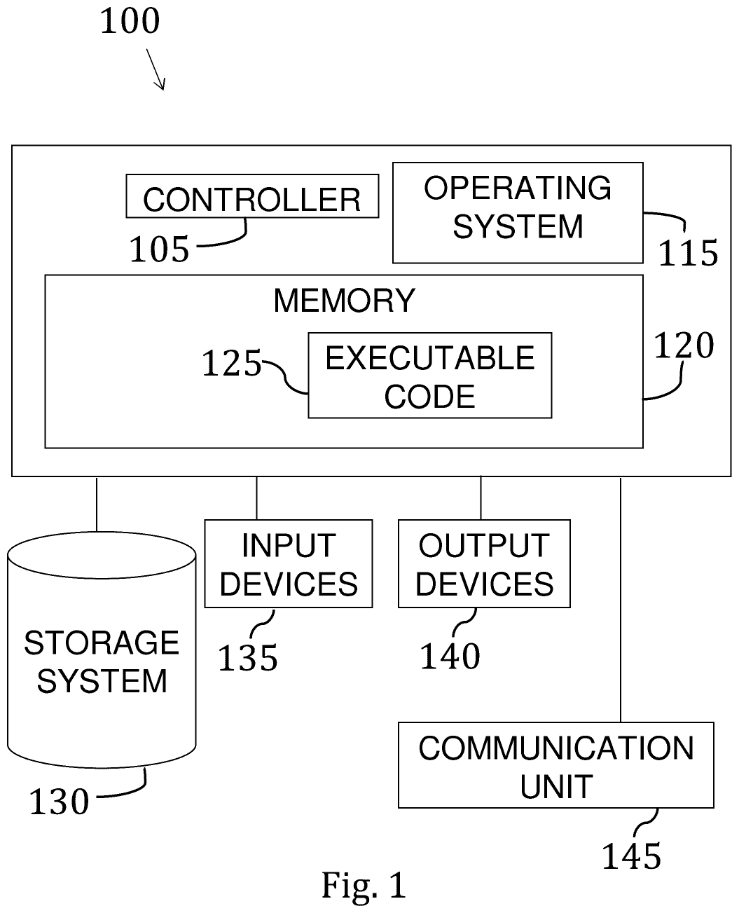

Reference is made to FIG. 1, which is a block diagram of an example computing device, according to some embodiments of the invention. Computing device 100 may include a controller or processor 105 (e.g., a central processing unit processor (CPU), a chip or any suitable computing or computational device), an operating system 115, memory 120, executable code 125, storage 130, input devices 135 (e.g. a keyboard or touchscreen), and output devices 140 (e.g., a display), a communication unit 145 (e.g., a cellular transmitter or modem, a Wi-Fi communication unit, or the like) for communicating with remote devices via a communication network, such as, for example, the Internet or using a near field communication (NFC) sensor.

Controller 105 may be configured to execute program code to perform operations described herein. The system described herein may include one or more computing device(s) 100. For example, system 200 for remote monitoring for ambulatory patients may be, or may include computing device 100 or components thereof.

Operating system 115 may be or may include any code segment (e.g., one similar to executable code 125 described herein) designed and/or configured to perform tasks involving coordinating, scheduling, arbitrating, supervising, controlling or otherwise managing operation of computing device 100, for example, scheduling execution of software programs or enabling software programs or other modules or units to communicate.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of similar and/or different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of operating system 115. For example, executable code 125 may be a software application that performs methods as further described herein.

Although, for the sake of clarity, a single item of executable code 125 is shown in FIG. 1, a system according to embodiments of the invention may include a plurality of executable code segments similar to executable code 125 that may be stored into memory 120 and cause controller 105 to carry out methods described herein.

Storage 130 may be or may include, for example, a hard disk drive, a universal serial bus (USB) device or other suitable removable and/or fixed storage unit. In some embodiments, some of the components shown in FIG. 1 are omitted. For example, memory 120 may be a non-volatile memory having the storage capacity of storage 130. Accordingly, although shown as a separate component, storage 130 may be embedded or included in memory 120.

Input devices 135 may be or may include a keyboard, a touch screen or pad, a camera one or more sensors or any other or additional suitable input device. Any suitable number of input devices 135 may be operatively connected to computing device 100. Output devices 140 may include one or more displays or monitors and/or any other suitable output devices. Any suitable number of output devices 140 may be operatively connected to computing device 100.

Any applicable input/output (I/O) devices may be connected to computing device 100 as shown by blocks 135 and 140. For example, a wired or wireless network interface card (NIC), a universal serial bus (USB) device or external hard drive may be included in input devices 135 and/or output devices 140.

Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium such as memory 120, computer-executable instructions such as executable code 125 and a controller such as controller 105.

Such a non-transitory computer readable medium may be for example a memory, a disk drive, or a USB flash memory, encoding, including or storing instructions, e.g., computer-executable instructions, which when executed by a processor or controller, carry out methods disclosed herein.

The storage medium may include, but is not limited to, any type of disk including, semiconductor devices such as read-only memories (ROMs) and/or random-access memories (RAMs), flash memories, electrically erasable programmable read-only memories (EEPROMs) or any type of media suitable for storing electronic instructions, including programmable storage devices. For example, in some embodiments, memory 120 is a non-transitory machine-readable medium.

In some embodiments, a system may include or may be, for example, a personal computer, a desktop computer, a laptop computer, a workstation, a server computer, a network device, or any other suitable computing device.

A system according to embodiments of the invention may include components such as, but not limited to, a plurality of central processing units (CPUs), a plurality of graphics processing units (GPUs), or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components.

In some embodiments, the terms “user”, “patient” and “consumer” are used interchangeably herein. The terms “external object” and “trigger” may also be used interchangeably herein.

According to some embodiments, a patient may use the capability of a mobile device to sense their environment to automatically report a health event to a remote monitoring system without the need to run a mobile application and/or manually enter the event data and without the need to use a special purpose medical device. As an example, users can hold their mobile device in proximity to a special purpose tag (e.g., NFC tag) with a single “wave” or “tap” to report a health event that otherwise would require them to make an effort to locate an app, locate the appropriate menu inside the app and/or then enter data-when they are most vulnerable and/or compromised after a health event such as a migraine attack. In some embodiments, the gathered data may be used to automatically alert a physician to alter the patient's medication when the number of reported seizures exceeds a predetermined threshold so a prescription for a new medication may also be automatically issued to the patient.

The tag may be externally affixed to any surface or carried by the patient in a convenient enclosure. The tag may not disclose its purpose to keep patient's privacy. Similarly, external triggers for automatic data reporting can be pre-set QR codes, pre-printed text shown to a camera or even distinctive visual objects around the user, for instance being pre-set in a mobile phone application for a specific use by a specific patient. All is to enable a user who experienced a health event to log the health event easily and/or promptly and/or securely making the data more accurate and/or useful for self-assessment or for later review by a healthcare professional for providing better care to the user, or for automatic assistance to the patient such as call to a caregiver when a seizure event is registered.

A health monitoring system may include: a mobile device for electronically recording an interaction with an external object or trigger, a sensing module embedded in the mobile device and/or adopted for sensing the object or objects used, a logging system in the mobile device that keeps data obtained from the external object, a decoding module for associating information derived from the tag with user's identity, and/or a communication module for communication with a remote storage system for the delivery of the event information to a remote care team.

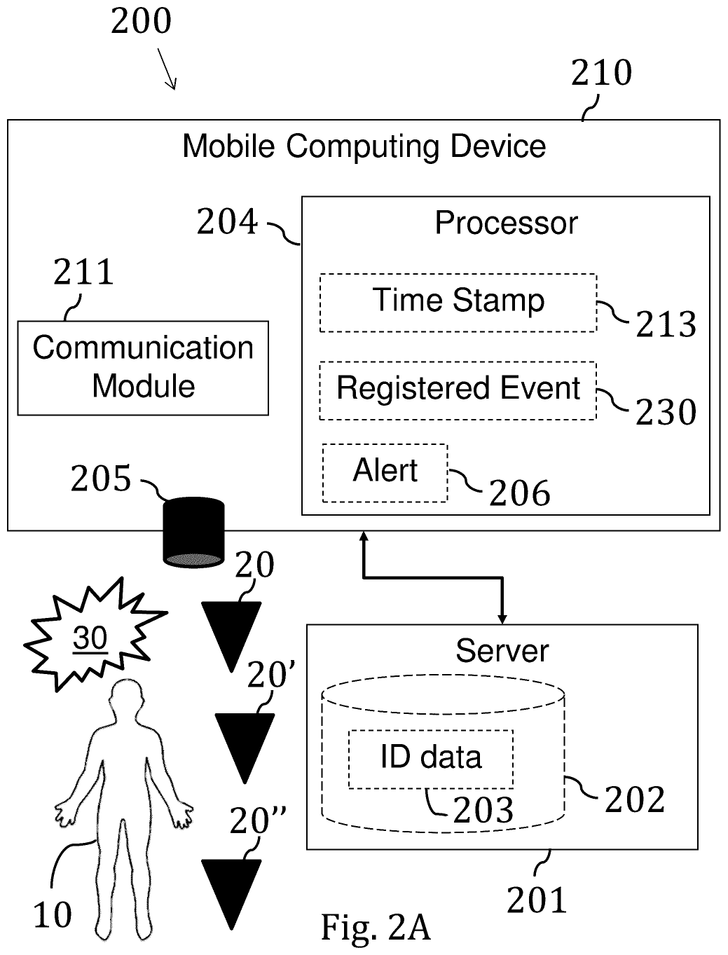

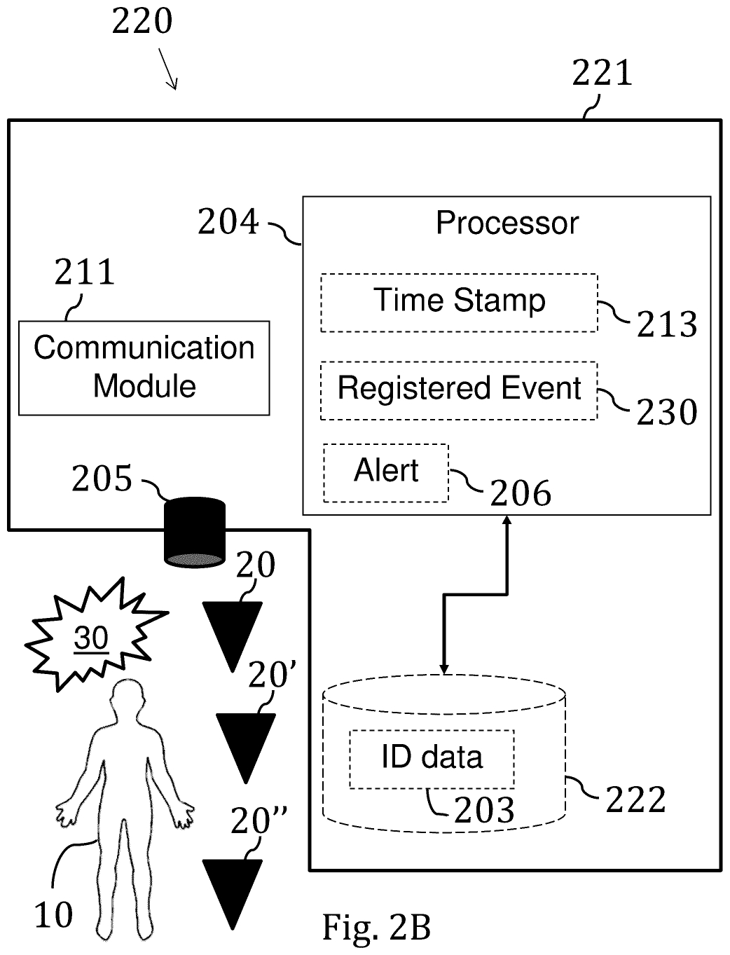

Reference is now made to FIG. 2A, which shows a block diagram for a system 200 for remote monitoring for an ambulatory patient 10, according to some embodiments of the invention. In FIG. 2A, some hardware elements may be indicated by a solid line while some software elements may be indicated by a dashed line.

The system 200 may include a remote server 201 with a database 202 of identification data 203 for a plurality of objects 20 associated with at least one event 30 by the patient 10.

The system 200 may include a processor 204 (e.g., such as the controller 105 shown in FIG. 1) of a mobile computing device 210 (e.g., such as the computing device 100 shown in FIG. 1), with the mobile computing device 210 being in communication with the remote server 201. For example, the mobile computing device 210 may communicate with the remote server 201 via wireless communication (e.g., via the Internet).

The mobile computing device 210 may include a communication module 211 for communication with the server 201.

In some embodiments, the processor 204 may verify the identification data 203 for at least one object 20′ of the plurality of objects 20 with the received database 202, when the mobile computing device 210 engages at least one object 20′ of the plurality of objects 20. Engaging the at least one object 20′ may include determining the identification data 203 with a time stamp 213. Thus, events may also be monitored based on the registered time stamp 213.

In some embodiments, the processor 204 may apply a time delay between consecutive registering of events to prevent mis identification of events. The system 200 for remote monitoring for an ambulatory patient 10 may apply, as an example, at least one algorithm to prevent multiple unintended taps when using NFC tags.

Repetitive or unintentional contact with the NFC tag by the patient, whether accidental or in an effort to confirm event logging, may result in data clutter and patient confusion. To mitigate this, a debouncing algorithm may be employed to introduce a set time delay (for example, one minute) following an initial interaction, during which further interactions with the same tag may be disregarded. Such a mechanism may be crucial to prevent duplicate actions, unintended logging, and/or erroneous data entries.

For example, the mobile computing device 210 may include an imager 205 or sensor to sense the at least one object 20′. The at least one object 20′ of the plurality of objects 20 may be an object that is external to the patient 10 and/or the mobile computing device 210, for instance the object may be a painting, a drawing, a landscape, a QR-code or barcode or any other visually distinctive object. Once the mobile computing device 210 engages and/or detects this tag or object, using the imager 205 or sensor, the processor 204 may accordingly verify the identification data associated with the tag.

Once the identification object or data stored on the object is verified by the processor, the processor 204 may register an event 230 by the patient 10 that is associated with the at least one object 20′. Either immediately or at a later time, the registered event 230 data may be sent to the server 201 and from there in some embodiment, to medical records of the patient and/or to be reviewed by a caregiver or physician.

In some embodiments, an alert 206 associated with the registered event 230 may be sent to a medical sever in communication with the mobile computing device 210 to register the event 30 for that patient 10. The alert 206 may be issued when a rate of registering events exceeds a predefined threshold (e.g., one event per minute), and a corresponding request may be automatically sent for a new prescription of a medication, when the registered event is for taking the medication (e.g., when many events cause reduction in amount of medication). For example, the request for the new prescription may be transmitted automatically to an electronic prescription refill service which automatically sends the prescription via courier and/or mail to the patient.

In some embodiments, in order to associate an event 30 (e.g., the patient experiencing a migraine or seizure) with a new object 20″, the patient 10 may use a dedicated software to register the new object 20″ with the event 30.

Reference is now made to FIG. 2B, which shows a block diagram for a system 220 for remote monitoring for the ambulatory patient 10, according to some embodiments of the invention. In FIG. 2B, some elements may be identical to similar elements described in FIG. 2A having the same numerals.

The system 220 may include a processor 204 (e.g., such as the processor 204 shown in FIG. 2A) of a mobile computing device 221. The mobile computing device 221 may include a memory with a database 222 of identification data 203 for a plurality of objects 20 associated with at least one event 30 by the patient 10.

The processor 204 may verify the identification data 203 for at least one object 20′ of the plurality of objects 20 with the received database 222, when the mobile computing device 221 engages at least one object 20′ of the plurality of objects 20. Engaging the at least one object 20′ may include determining the identification data 203 with a time stamp 213. Thus, events may also be monitored based on the registered time stamp 213.

Some embodiments described for elements in FIG. 2A may be applied to similar elements in FIG. 2B, where the database is stored on the mobile computing device instead of being received from the remote server.

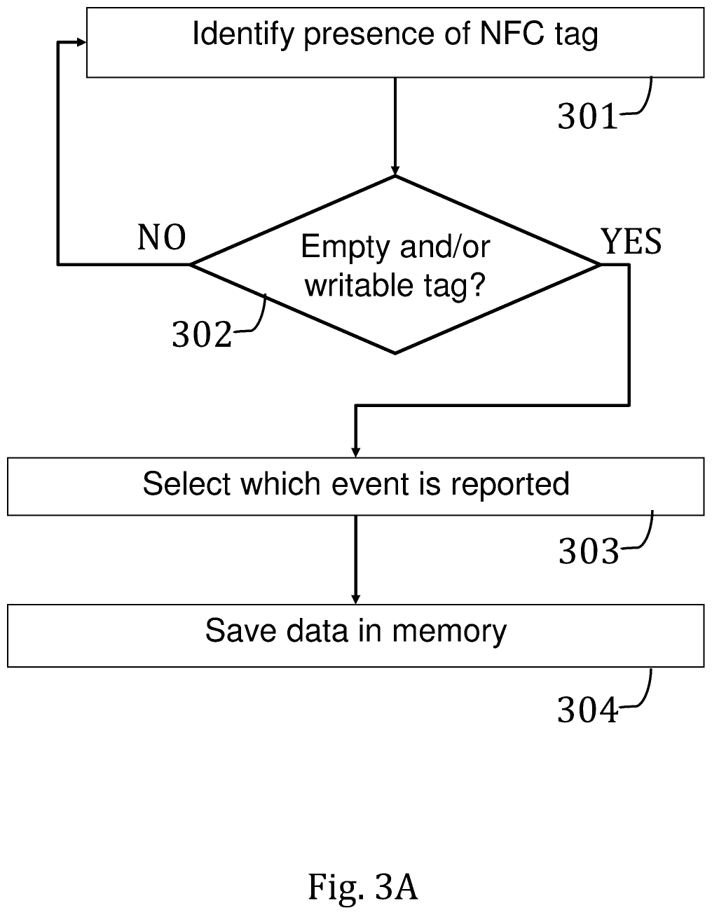

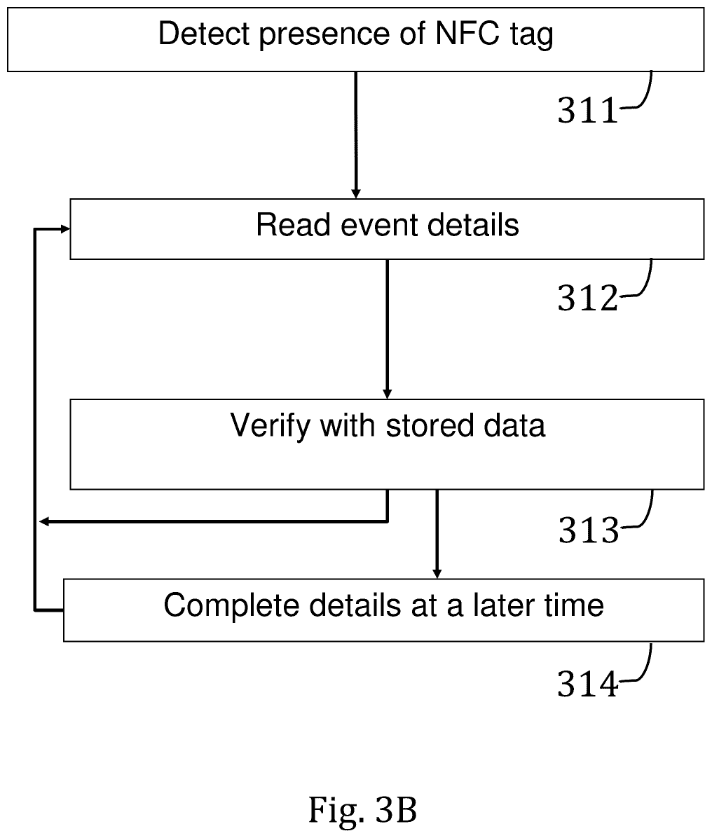

Reference is now made to FIG. 3A, which shows a flowchart for associating an event with NFC tags, according to some embodiments of the invention.

The new object 20″ (as shown in FIG. 2A or 2B) may be identified 301 (e.g., by a smartphone) when presence of NFC tag is detected. If the tag is an empty and/or writable NFC tag 302 (e.g., placed near the patient's bed), the patient may select 303 which event is to be reported. Once the event is selected, the event data may be stored 304 in memory of the computing device that communicated with the NFC tag, and/or stored in memory of a remote server. If the NFC tag either has data or is not writable, an error message may be displayed on a user interface display (e.g., on a smartphone) giving the patient 10 the option to replace that tag until a new NFC tag may be identified.

For example, the patient 10 (as shown in FIG. 2A or 2B) may use the dedicated software of a mobile application on a smartphone to identify a new object 20″ and/or register that object with a selected event 30 (e.g., the event may be for experiencing a migraine or seizure). The patient 10 may be instructed (e.g., by the mobile application) to hold the smartphone in vicinity to the NFC tag for a few seconds, until the NFC tag is detected by the smartphone. The patient 10 may then be instructed (e.g., by the mobile application) to name this tag and/or to select the event that should be registered by identifying this NFC tag at a later stage. Accordingly, the mobile application may provide a list of registered objects and their associated events for the patient 10 to monitor and/or edit.

Reference is now made to FIG. 3B, which shows a flowchart for reporting an event with NFC tags, according to some embodiments of the invention.

In some embodiments, an NFC tag may be engaged and/or detected 311 by the mobile computing device 210 (such as a smartphone) at an arbitrary point in time. Then unique identifying data (associated with the event) may be accordingly read 312 from the NFC tag by the mobile computing device 210. The unique identifying data may be verified 313 and/or matched with the ID data 203 in the server 201 and/or matched with data stored on the mobile computing device 210. For example, the patient 10 may be asked to acknowledge reporting an event data as pre-set, to avoid an accidental reporting.

Some details concerning the event (e.g., severity of pain, duration of the event, or precise timing of the event) may be completed 314 at a later time after the event is over, and prompted to the user. In some embodiments, two separate objects (e.g., two NFC tags) may be defined as a pair, one for the beginning of the event and one for the end, thereby allowing an easy and more accurate determination of the event duration.

In some embodiments, the new object 20″ may be a barcode or QR code that may be detectable by the imager 205 (e.g., built in a smartphone).

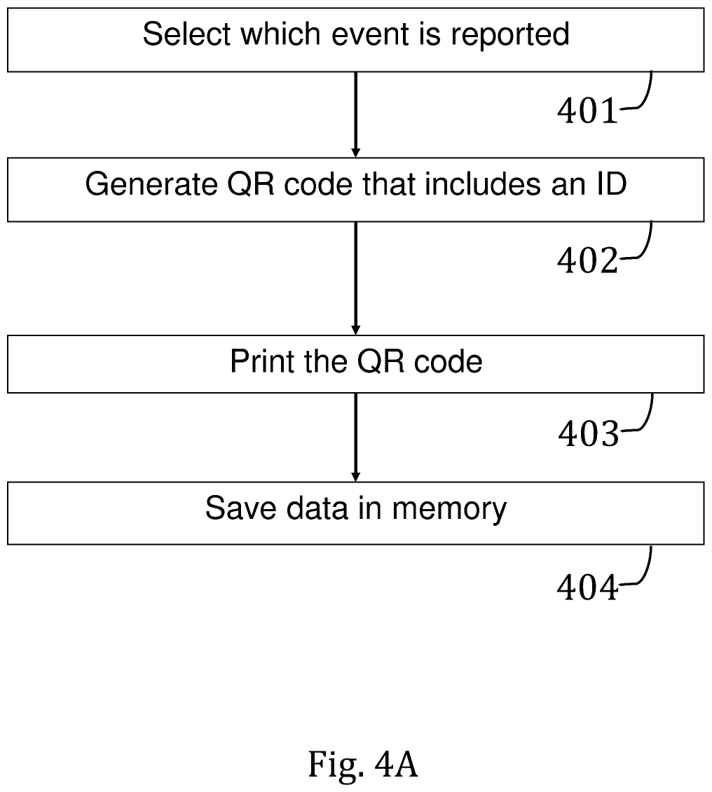

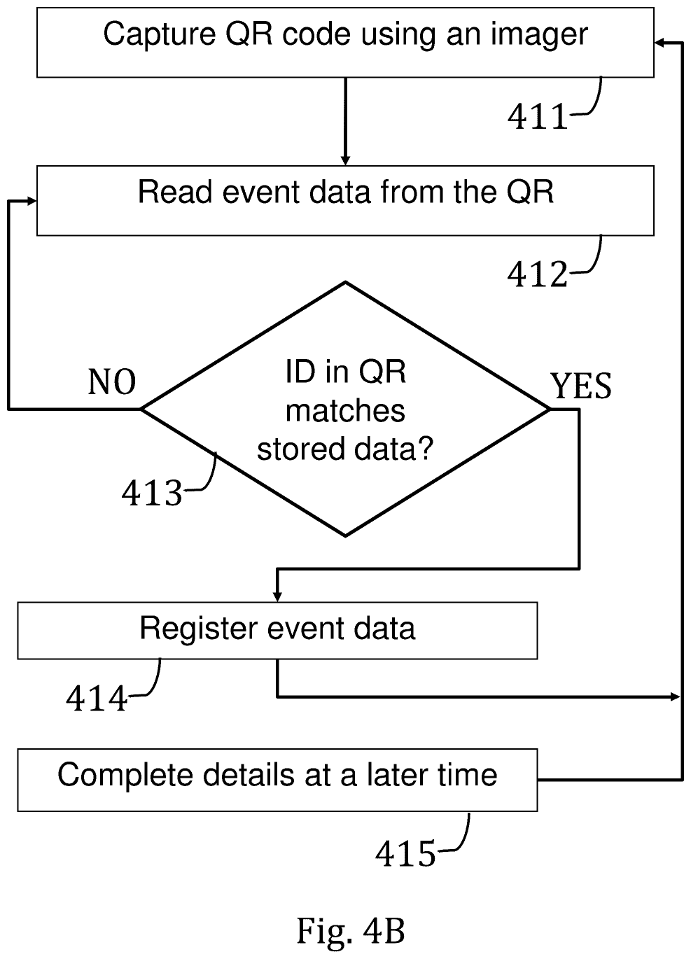

Reference is now made to FIG. 4A, which shows a flowchart for associating an event with a QR code, according to some embodiments of the invention.

The patient 10 (as shown in FIG. 2A or 2B) may create dedicated barcodes or QR codes by selecting 411 an event 30 (e.g., a migraine) and generating 412 the dedicated barcodes or QR codes (e.g., printing 413 in a printer and positioning at a desired location) that are associated with the selected event. The event data may be stored 414 in memory of the computing device that captured the QR code, and/or stored in memory of a remote server. The next time that the selected event 30 (e.g., a migraine) occurs, the patient 10 may engage the object by capturing (e.g., by a camera of a smartphone) the generated barcodes or QR codes so as to register the event 30.

Reference is now made to FIG. 4B, which shows a flowchart for reporting an event with QR codes, according to some embodiments of the invention.

The patient 10 may engage the object by capturing 411 (e.g., by a camera) the generated barcodes or QR codes such that identification data in the generated barcodes or QR codes may be read 412 to extract an identification number or identification code. That identification data may be compared to the pre-set ID data 203 in server 201 (and/or matched with data stored on the mobile computing device 210) and once a match is found 413, relevant event data may be registered 414. For example, the patient 10 may be asked to acknowledge reporting an event data as pre-set, to avoid an accidental reporting.

Some details concerning the event may be completed 415 at a later time after the event is over, and prompted to the user. For example, these details may include at least one of: severity of pain, duration of the event, precise timing of the event, pain position on the body, type of a seizure, sleep quality, sleep duration, medication side effects (e.g., a rush, a headache, sleepiness, or irritated stomach), and/or extra medication taken. In some embodiments, two separate objects (e.g., two QR codes) may be defined as a pair, one for the beginning of the event and one for the end, thereby allowing an easy and more accurate determination of the event duration.

In some embodiments, a dedicated wearable health monitoring device of the patient may detect a health event and automatically register the health event with an adjacent NFC tag.

According to some embodiments, the new object 20″ may be provided with predefined data, for instance the new object 20″ is provided as already associated to a specific event, such that there may be no need for registering the new object 20″ with the dedicated software.

In some embodiments, the new object 20″ may be any object selected by the patient 10.

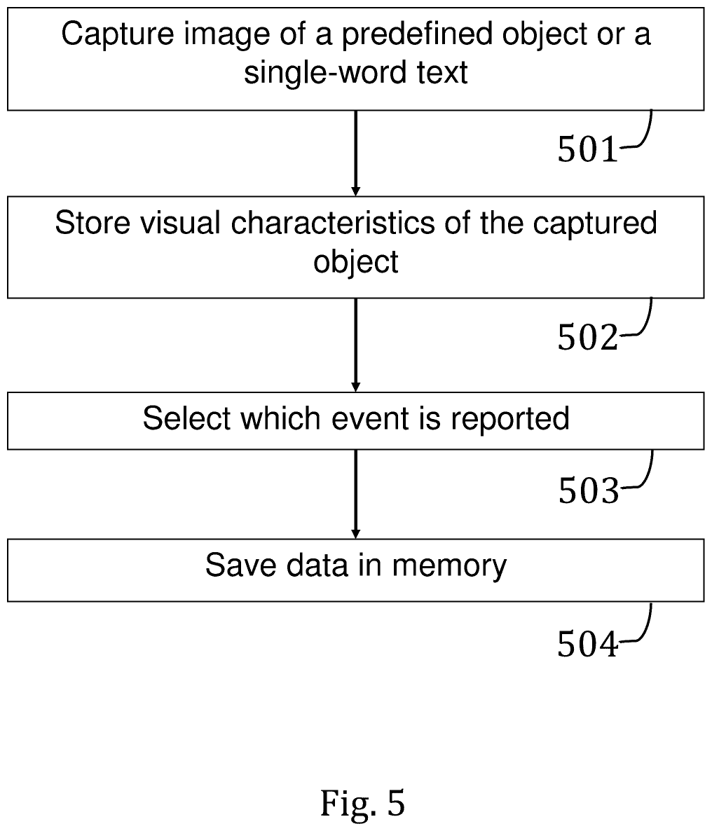

Reference is now made to FIG. 5, which shows a flowchart for associating an event with a predefined object, according to some embodiments of the invention.

For example, the patient may select the object as a painting on a wall at the bedroom of the patient, a bold letter text (e.g., a single word in large font for visually impaired patients) near the medicine cabinet of the patient 10 (as shown in FIG. 2A or 2B), or a special piece of furniture or a household item such as a plant in the patient's kitchen. By initially registering the selected object(s) to be associated with an event 30, and then capturing 501 the selected object(s), for instance by the imager 205, to store 502 visual characteristics of the captured object(s) for a selected event 503, the patient 10 may automatically register the associated event 30. Thus, automatic events may be registered instead of the patient 10 be required to click and use software which may be difficult for some patients. The event data may be stored 504 in memory of the computing device that captured the selected object(s), and/or stored in memory of a remote server.

For example, image processing algorithms may be used to identify the selected object(s) from images captured by a camera operated by the patient 10. The patient 10 may be instructed (e.g., by the mobile application) to direct the imager 205 towards the selected object and capture at least one image to register that new object.

According to some embodiments, when a new object 20″is registered to be associated with an event, dedicated and/or unique identifying data may be embedded into the object (e.g., in case of NFC tag or QR code) and may also be saved in the memory of the mobile computing device 210. For example, the dedicated and/or unique identifying data may include a unique identification number and/or a string of characters.

The unique identifying data may be generated by the processor of the mobile computing device, and the event may be accordingly associated to a new object by the generated unique identification data.

Once the new object 20″ is engaged again, the identification data of that object may be compared with other objects defined in the mobile computing device 210 and/or the server 201. Only if there is a match to the dedicated and/or unique identifying data of that object, the event reporting may occur. This may prevent other users from accidentally trigger data reporting by having their engaging their mobile computing devices to objects associated with someone else.

For example, the dedicated and/or unique identifying data may be generated by the server 201 and/or by the processor 204. The dedicated and/or unique identifying data may be stored in the non-volatile memory or data storage available in the mobile computing device 210 and/or the server 201.

Referring back to FIG. 2A. According to some embodiments, system 200 for remote monitoring for an ambulatory patient 10 may apply at least one algorithm to prevent different patients from registering the same event unintentionally.

A security vulnerability and/or potential for patient confusion may arise if patient A sets and/or engages an object (e.g., NFC tag) using a mobile computing device A to register an even occurring to patient A, and later patient B using the same mobile application on their mobile computing device B engages the same object with their mobile computing device B. If the mobile computing device B recognizes that object, it may unintentionally log a wrong event, leading to inaccurate data and user confusion.

To mitigate unintentional event registering, a combination of unique data identifiers and/or access controls within the mobile application may be applied. A unique multi-digit random identifier, as the unique data ID, may be generated and/or written for the object at the time of its initial setup by patient A. For example, the unique data ID may include a 16-digit number. The generated unique data ID may be stored in the memory of the mobile application on the mobile computing device A for future identification and verification purposes.

Upon subsequent engagement of the object by a mobile computing device, the processor and/or the mobile application may initiate a comparison sequence by reading the unique data ID from the detected object and matching it against the stored IDs within the memory. If a match is identified, the mobile application may log the corresponding event. If no match is found, such as in cases where an unintended user, patient B, engages the tag, the processor and/or the mobile application may disregard the object, thereby mitigating the risk of erroneous data entry.

For example, the unique data ID, may be a composite identifier including a timestamp represented in Unix format, combined with a unique user identifier associated with each user, and an additional randomly generated number to complete the 16-digit number.

In some embodiments, the processor may apply a one-function object limit, enabling users to assign multiple objects, each dedicated to a distinct data item. Each object may be allocated to a specific event and/or data category, such as headache occurrence, medication intake, or sleep tracking. Thus, the system allows organized and/or discrete data capture, ensuring each object serves a unique function and minimizes the potential for data overlap or redundancy across multiple events.

In some embodiments, the processor of the mobile computing device may store identification data for a plurality of objects on a dedicated server (e.g., a dedicated medical server or server 201) and accordingly recover the identification data from the dedicated server, when the mobile computing device is not available (e.g., when the mobile device is lost or due to a malfunction).

According to some embodiments, system 200 for remote monitoring for an ambulatory patient 10 may apply at least one algorithm to prevent data loss when the mobile computing device is no longer available (e.g., when lost, broke, or replaced).

In cases where a mobile computing device is lost, stolen, damaged, or replaced with a different brand, locally stored data may be difficult to transfer. To retain object information and assignments, essential object data may be stored in a dedicated server (e.g., a dedicated medical server or server 201). For example, the object data may be stored along with the object's hard-coded unique ID, generated during the setup phase, and the assignment to events associated with each object. This server-based storage may enable restoration of object data and/or assignments on a new or replacement device. Additionally, the object's physical characteristics, such as color or any printed letters, may also be stored on the server as an optional feature, allowing for complete restoration of object appearance and configuration if needed.

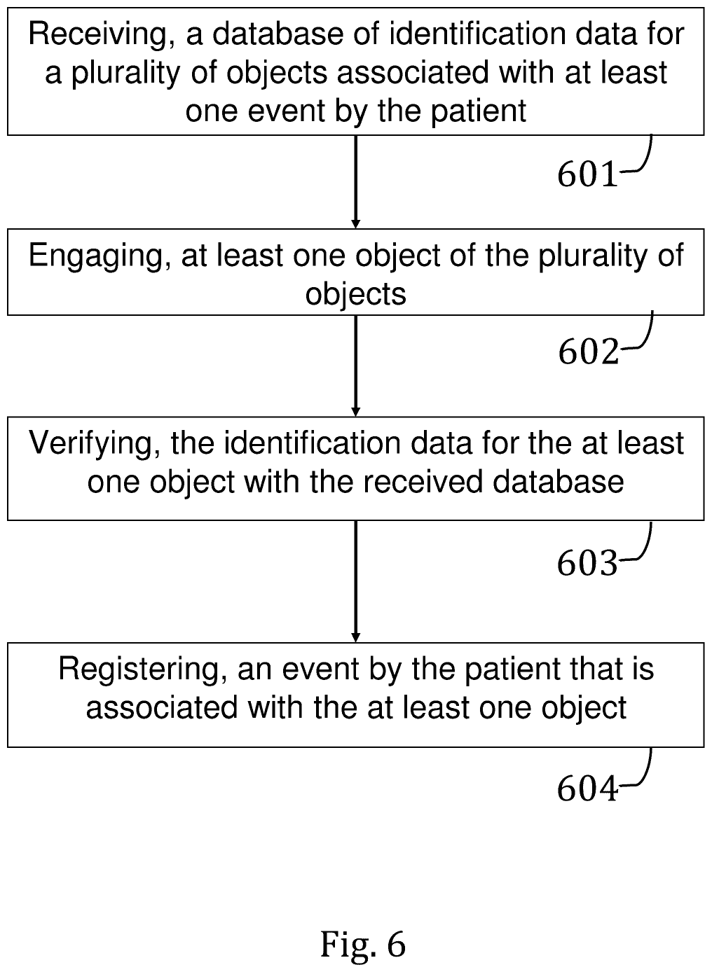

Reference is now made to FIG. 6, which shows a flowchart for a method of remote monitoring for an ambulatory patient, according to some embodiments of the invention.

A database of identification data may be received 601 (by a processor of a mobile computing device) for a plurality of objects associated with at least one event by the patient.

At least one object of the plurality of objects may be engaged 602 by the mobile computing device.

The identification data for the at least one object may be verified 603 (by the processor of the mobile computing device) with the received database, and an event may be registered 604 (by the processor of the mobile computing device) by the patient that is associated with the engaged object.

While certain features of the invention have been illustrated and described herein, many modifications, substitutions, changes, and equivalents may occur to those skilled in the art. It is, therefore, to be understood that the appended claims are intended to cover all such modifications and changes.

Various embodiments have been presented. Each of these embodiments may of course include features from other embodiments presented, and embodiments not specifically described may include various features described herein.

1. A method of remote monitoring for an ambulatory patient, the method comprising:
receiving, by a processor of a mobile computing device, a database of identification data for a plurality of objects associated with at least one event by the patient;
engaging, by the mobile computing device, at least one object of the plurality of objects;
verifying, by the processor of the mobile computing device, the identification data for the at least one object with the received database; and
registering, by the processor of the mobile computing device, an event by the patient that is associated with the engaged object.

2. The method of claim 1, wherein registering the event comprises sending an alert to a medical sever in communication with the mobile computing device to register the event for that patient.

3. The method of claim 1, wherein engaging the at least one object comprises determining the identification data with a time stamp.

4. The method of claim 3, wherein engaging the at least one object is carried out by a wireless communication module of the mobile computing device.

5. The method of claim 3, wherein engaging the at least one object is carried out by an imager of the mobile computing device.

6. The method of claim 5, further comprising recording a new object by the imager of the mobile computing device.

7. The method of claim 1, further comprising:
issuing an alert when a rate of registering events exceeds a predefined threshold; and
sending a request for a new prescription of a medication, wherein the registered event is for taking the medication.

8. The method of claim 1, further comprising applying, by the processor of the mobile computing device, a time delay between consecutive registering of events.

9. The method of claim 1, further comprising:
generating, by the processor of the mobile computing device, a unique identification code; and
associating, by the processor of the mobile computing device, an event to a new object by the generated unique identification code.

10. The method of claim 1, further comprising:
storing, by the processor of the mobile computing device, identification data for a plurality of objects on a dedicated server; and
recovering the identification data from the dedicated server, when the mobile computing device is not available.

11. A system for remote monitoring for an ambulatory patient, the system comprising:
a remote server with a database of identification data for a plurality of objects associated with at least one event by the patient; and
a processor of a mobile computing device, in communication with the remote server, wherein the processor is configured to:
verify the identification data for at least one object of the plurality of objects with the received database, when the computing device engages the at least one object of the plurality of objects; and
register an event by the patient that is associated with the engaged object.

12. The system of claim 11, wherein the processor is further configured to send an alert to a medical sever in communication with the mobile computing device to register the event for that patient.

13. The system of claim 11, wherein the processor is further configured to determine the identification data with a time stamp, when the computing device engages at least one object.

14. The system of claim 13, wherein the mobile computing device comprises a wireless communication module to engage the at least one object.

15. The system of claim 13, wherein the mobile computing device comprises an imager to engage the at least one object.

16. The system of claim 15, wherein the processor is further configured to record a new object by the imager of the mobile computing device.

17. The system of claim 11, wherein the processor is further configured to:
issue an alert when a rate of registering events exceeds a predefined threshold; and
send a request for a new prescription of a medication, wherein the registered event is for taking the medication.

18. The system of claim 11, wherein the processor is further configured to:
generate a unique identification code; and
associate an event to a new object by the generated unique identification code.

19. The system of claim 11, wherein the processor is further configured to:
store identification data for a plurality of objects on the remote server; and
recover the identification data from the remote server, when the mobile computing device is not available.

20. A system for remote monitoring for an ambulatory patient, the system comprising:
a processor of a mobile computing device, comprising:
a database of identification data for a plurality of objects associated with at least one event by the patient;
wherein the processor is configured to:
verify the identification data for at least one object of the plurality of objects with the database, when the computing device engages the at least one object of the plurality of objects; and
register an event by the patient that is associated with the at least one object.
